(19) United States
(12) Patent Application Publication
(10) Pub. No.: US 2023/0278017 A1
(43) Pub. Date: Sep. 7, 2023

(54) MFI ZEOLITE OF HIGHLY DISPERSED FRAMEWORK ALUMINUM AND ITS USES FOR SELECTIVE AROMATICS METHYLATION TO PARA-XYLENE

(71) Applicant: UOP LLC, Des Plaines, IL (US)

(72) Inventors: Deng-Yang Jan, Elk Grove Village, IL (US); Rajamani Gounder, West Lafayette, IN (US); Sopuruchukwu Ezenwa, West Lafayette, IN (US); Jaime G. Moscoso, Mount Prospect, IL (US); Gregory Kuzmanich, Arlington Heights, IL (US)

(21) Appl. No.: 18/176,465

(22) Filed: Feb. 28, 2023

Related U.S. Application Data
(60) Provisional application No. 63/315,429, filed on Mar. 1, 2022.

(51) Int. Cl. B01J 29/70; C07C 2/66; B01J 29/76

(52) U.S. Cl. CPC B01J 29/7049; C07C 2/66; B01J 29/76; C07C 2529/76

(57) ABSTRACT

A process for contacting a feed stream comprising an oxygenate feedstock and an aromatic feedstock comprising toluene with a catalyst and recovering a product comprising para-xylene. The catalyst comprises an improved MFI zeolite comprising in the calcined and ion-exchanged form a SiO₂/Al₂O₃ ratio of from about 50 to about 600 and having a distribution of framework aluminum sites characterized by an initial xylene selectivity of greater than 70% in the TM diagnostic test.

### CROSS-REFERENCE TO RELATED APPLICATIONS

This application claims priority from U.S. Provisional Application No. 63/315,429, filed Mar. 1, 2022, which is incorporated herein in its entirety.

### FIELD

The field is production of para-xylene and includes a process comprising contacting toluene and an oxygenate over a MFI zeolite catalyst.

### BACKGROUND

MFI zeolite is a versatile catalyst for carrying out a wide array of catalytic processes for petrochemical production, such as converting oxygenates to light olefins in methanol to propylene) and methanol to aromatics, or to co-produce light olefin and aromatics.

Typically, an Aromatics Complex is designed to co-produce xylene and benzene with xylene being the primary product. Among xylene isomers, para-xylene makes up the majority of demand for xylene product. The amount of xylene relative to benzene is pre-determined by the methyl to phenyl ratio of the reformate coming into Aromatics Complex, which is derived, for example, from a reforming process. The overall methyl to phenyl ratio in an aromatics complex is typically less than 2.0. The methyl to phenyl ratio is required to be 2.0 to have 100% para-xylene production and net-zero benzene production. One approach considered commercially to maximize para-xylene production is to methylate toluene and benzene using oxygenates such as methanol to increase methyl to phenyl ratios towards 2.0. Methyl to phenyl ratio may be calculated by dividing the number of methyl groups by the number of aromatic centers for the entire product. For example, benzene has 0 methyl groups and 1 phenyl moiety giving a methyl/phenyl ratio of 0. Toluene has 1 methyl group and 1 phenyl moiety and a methyl/phenyl ratio of 1. Each of the three xylenes has 2 methyls and 1 phenyl for a methyl/phenyl ratio of 2. Methyl to phenyl ratios of greater than 1 or greater than 1.5 or greater than 1.75 are desired. Further, in toluene methylation, it is highly desirable to perform the process to attain high xylene yields at very high para-xylene to xylene ratios of greater than, for example, 90%, to avoid difficult and costly para-xylene extraction process.

Typically, toluene methylation is operated to selectively produce para-xylene. Severe process conditions, namely high temperature, are used where methanol conversion to hydrocarbons (MTH) or gasoline (MTG) becomes increasingly significant and methanol decomposition to COₓ and H₂ is appreciable. Significant amounts of diluents such as H₂O, H₂ and thus recycle streams are used, rendering a catalyst relatively difficult to prepare reproducibly. MFI zeolite has been the catalyst used predominantly in this process.

As used herein, zeolites may be referred to by an improper name, such as silicalite, a proper name, such as ZSM-5, or by structure type code, such as MFI. These three letter codes indicate atomic connectivity and hence pore size, shape and connectivity for the various known zeolites. The list of these codes may be found in the Atlas of Zeolite Framework Types, which is maintained by the International Zeolite Association Structure Commission at http://www.iza-structure.org/databases/. At present, 255 structure types are known and catalogued by the IZA. One such structure type, MFI is described as comprising 3-dimensional 10-ring channels with straight channels along crystallographic axis b and tortuous channels along axis a. Zeolites are distinguished from each other on the basis of their composition, crystal structure, and adsorption properties. One method commonly used in the art to distinguish zeolites is x-ray diffraction.

The toluene methylation process is carried out in large part by Zeolite MFI. To achieve very high para-xylene purity in xylenes, zeolite MFI is typically “selectivated” to attain a shape selective effect to favor para-xylene molecule production. The primary means to achieve “selectivation” is via methods such as deposition of SiO₂ using silicon containing compounds, alkali earth oxide such as MgO, phosphate and a combination of the aforementioned as shown in U.S. Pat. No. 6,504,072. The chemical deposition step is regularly followed by steaming of varying degrees to further the shape selective effect. Such “selectivation” treatments are aimed to neutralize the external acidity and to constrain the zeolite pore mouths to a degree that allows para-xylene to selectively diffuse out of microporous pores, while restricting both meta- and ortho-xylene from coming out of the micropores. The selectivation procedure via conventional means is highly heterogeneous due to morphological heterogeneity of starting MFI material and the highly reactive nature of selectivating reagents with Zeolite MFI surfaces, with the outcome of selectivation being affected by many material variables and procedural parameters.

Selectivation processes may reduce oxygenate utilization. For example, the methanol utilization could drop below 60%, below 50% and even below 40% as the catalyst is modified to produce a para-xylene purity of 86-92, 97 and then 98% using a catalyst composition of phosphorus, ZSM-5 of 225 Si/Al ratio and a binder comprising silica alumina and clay as shown in U.S. Pat. No. 6,504,072.

Silicon compounds have also been used to selectivate an extrudate comprising ZSM-5 (Si/Al=13) and SiO₂ binder in US 2005/0143613, in which the effects of numbers of selectivation treatments, steaming, platinum incorporation, the sequence of platinum incorporation, steam and ZSM-5 Si/Al ratios were investigated. These variables have significant impacts on para-xylene purity, catalyst stability and methanol utilization only in the range of 40-50%. When employing ZSM-5 of Si/Al=225 and 1060° C. steaming, 60% methanol utilization may be achieved at 90% para-xylene purity.

Another approach used to attain para-xylene selectivity is via the use of zeolite MFI of specific morphology. Crystals comprising intergrown MFI wherein sinusoidal channels are exposed over 73% of crystallite exterior have been shown to result in enhanced para-xylene selectivity, Nature Communications, 2019, 10, 4348. Even under mild conditions favorable for para-xylene formation, i.e., atmospheric pressure, H₂O and H₂ co-feed and 470° C. using a feed of toluene to methanol molar ratio of 2.0 and ZSM-5 of 150 Si/Al ratio, only moderate toluene conversion of about 20% was achieved with low methanol utilization of about 50%.

Mass transport also needs be taken into account. Toluene methylation and subsequent xylene isomerization have been shown to be mass transport controlled in selectivated MFI (Ind. Eng. Chem. Res. 2017, 56, 9310-9321) when employing a caustic modified zeolite ZSM-5 and subsequent MgO selectivation. Methanol utilization is still low due to kinetically controlled methanol to hydrocarbons (MTH) side reactions. A drawback of improving mass transport properties by generating mesopores is the accompanied abundance of surface functional groups such as silanol, making surface passivation challenging.

While achieving high para-xylene purity via the aforementioned approaches, the consequence is the trade-off for significantly low methanol utilization (40-60%). Methanol utilization is defined (moles of xylene formed—moles of benzene formed)/(moles of methanol converted). The methanol utilization reflects the amount of oxygenates going to xylene as opposed to non-aromatics including olefinic and paraffinic hydrocarbons and to heavy aromatics. Due to an appreciable oxygenate cost, the techno-economics of aromatics methylation processes would vary with the oxygenate cost and the price differentiation of para-xylene versus benzene. Therefore, the viability and propagation of aromatics methylation to para-xylene would at minimum overcome the oxygenate utilization issue.

The temperature also affects the xylene selectivity during toluene methylation. Lower reaction temperatures (below about 300° C.) favor the formation of ortho-xylene over para-xylene and meta-xylene which is the intrinsic product selectivity typically observed on MFI zeolites, diminished effects of secondary reactions (such as xylenes isomerization, polymethylbenzene dealkylation, toluene disproportionation) and negligible xylenes diffusion restrictions. Low temperatures also favor methanol utilization because of significant reduction in rate of aromatic dealkylation.

Accordingly, it is desirable to provide improved methods and apparatuses for methylation of aromatic compounds such as toluene and benzene in an aromatics complex. Further, it is desirable to provide a cost-effective method for a high selectivity to para-xylene over a catalyst comprising an improved MFI zeolite for toluene and/or benzene methylation which operates under mild conditions, promotes high utilization of the feedstock and where higher than equilibrium pX/X ratios can be achieved without using dilution. Furthermore, other desirable features and characteristics of the present subject matter will become apparent from the subsequent detailed description of the subject matter and the appended claims, taken in conjunction with this background of the subject matter.

### BRIEF SUMMARY

Here we disclose the use of a catalyst comprising an improved MFI zeolite comprising altered distributions of acid sites for the production of para-xylene at high conversion through contacting a feed stream comprising toluene and an oxygenate. Generally, the improved MFI zeolite catalysts can be characterized as having low populations of proximate framework aluminum sites. The high dispersion of acid sites in MFI zeolites may also be associated with increasing fractions of acid sites located in the smaller channel pores relative to the larger channel intersections. The altered distributions of framework aluminum sites are further characterized by a performance in the TM diagnostic test of highly selective para-xylene formation under a condition of oxygenate (dimethyl ether, DME) to toluene molar ratio of 6 to 16 and a temperature of 130 C. The zeolite MFI is characterized by SiO₂/Al₂O₃ ranging from about 50 to about 600 and preferably by the incorporation of boron into the framework with SUB ranging from about 20 to about 50. The MFI zeolite used for selective aromatics methylation for para-xylene production may be synthesized using specific organic structural directing agent (OSDA) including ethylenediamine (EDA) and 1,4 diazobicyclo[2.2.2]octane (DABCO) with SiO₂/Al₂O₃ ratios ranging from 40 to 1000 and preferably having boron incorporated into the synthesis gel.

An intrinsically more selective MFI zeolite would enable higher para-xylene production throughput and lower production cost due to its significantly higher methanol utilization. Furthermore, when combining the intrinsically para-xylene selective MFI with a favorable morphology for aromatics methylation, an economically favorable process over the fluctuations of oxygenate feed cost and para-xylene and benzene price differentiation can be attained.

### BRIEF DESCRIPTION OF THE DRAWINGS

FIG. 1 is plot showing xylene isomer selectivity for the different MFI examples at 130° C., 4 kPa Toluene and 66 kPa dimethyl ether. A indicates the group of materials of Comparative Examples 2. B indicates the group of materials of Examples 1. E is Equilibrium distribution of xylene isomers at 130° C.

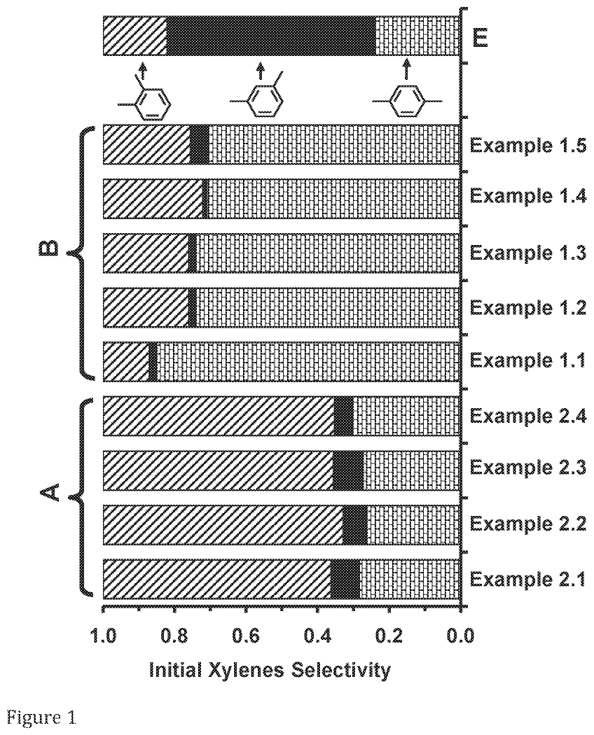

FIG. 2 is a plot showing xylenes isomer selectivity at varying toluene conversions (0.1-1.0%) at 130° C. during toluene methylation with dimethyl ether. Filled symbols are for Example 2.1 while open symbols are for Example 1.1. Para-xylene (diamonds), meta-xylene (circles), ortho-xylene (squares).

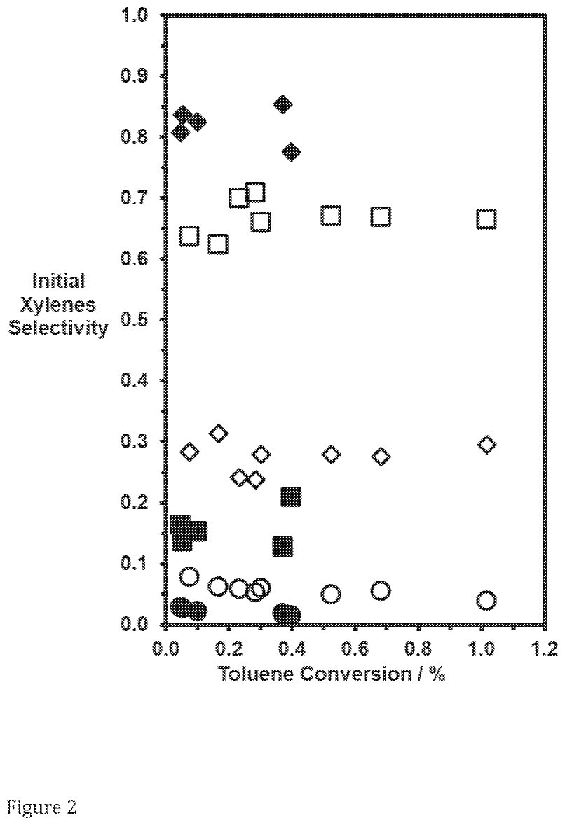

FIG. 3 is a plot showing xylenes isomer selectivity at varying toluene conversions (0.005-0.05%) at 130° C. during toluene methylation with methanol. Filled symbols are for Example 2.1 while open symbols are for Example 1.1. Para-xylene (diamonds), meta-xylene (circles), ortho-xylene (squares).

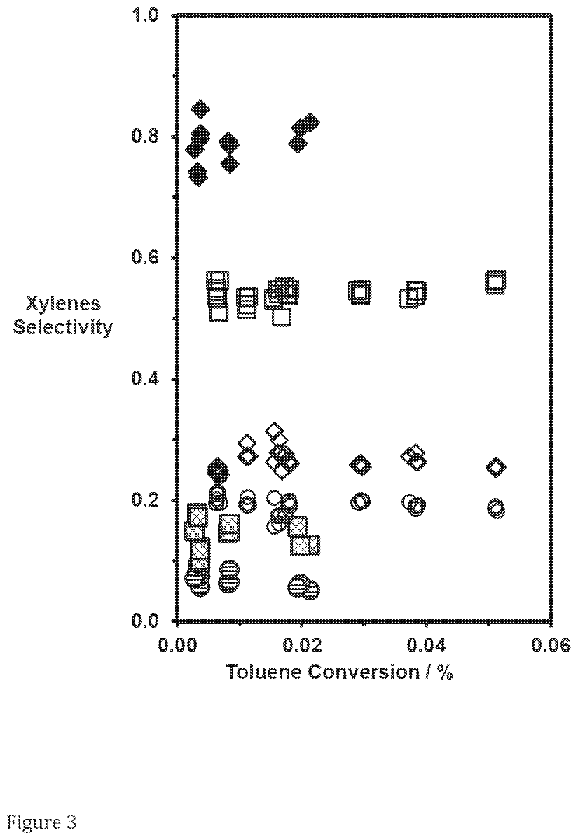

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

As used herein, the term “predominant” or “predominate” or “predominantly” means greater than 50%, suitably greater than 75% and preferably greater than 90%.

The term “Cx” is to be understood to refer to molecules having the number of carbon atoms represented by the subscript “x”. Similarly, the term “Cx−” refers to molecules that contain less than or equal to x and preferably x and less carbon atoms. The term “Cx+” refers to molecules with more than or equal to x and preferably x and more carbon atoms.

As used herein, the terms “xylene” or “xylenes” describe the class of dimethyl benzene molecules comprising one or more of 1,2-dimethylbenzene, 1,3-dimethylbenzene, and 1,4-dimethylbenzene. 1,2-dimethylbenzene is often referred to as ortho-xylene or oX. 1,3-dimethylbenzene is often referred to as meta-xylene or mX. 1,4-dimethylbenzene is often referred to as para-xylene or pX.

### DETAILED DESCRIPTION

The disclosure provides a process for producing para-xylene comprising contacting a feedstream with an improved zeolite MFI that is intrinsically more para-xylene selective. By combining a favorable morphology with a favorable distribution of framework aluminum sites characterized by particular reactivity in the TM diagnostic test, the selective aromatics methylation for para-xylene production may be accomplished with high para-xylene purity out of total xylene and high oxygenate utilization.

The objective of selective aromatics methylation is to achieve high para-xylene purity and high oxygenate utilization. As described in the background, the conventional means to achieve high para-xylene purity is via selectivation procedures. Oxygenates used in the process for the production of para-xylene may comprise methanol, dimethyl ether, dimethyl carbonate or a mixture of thereof.

The aforementioned trade-off of methanol utilization with para-xylene purity is inherent in that the conversion of methanol to side products, such as light olefins and paraffins, goes in part through poly-methylated intermediates in the MTH chemistry. Zeolite MFI prepared via conventional OSDA such as tetrapropylammonium (TPA) is shown to produce bulkier ortho-xylene isomer and consecutive methylated product such as trimethylbenzene under the prescribed diagnostic conditions of 130° C. and DME to toluene molar ratio of 16 as shown in comparative examples described below. This reactivity pattern would lead the reactions down the pathway of light olefin and paraffin formation, and thus lower methanol utilization. This reactivity pattern would also require secondary isomerization reactions of the ortho-xylene isomer to obtain desired para-xylene.

Depending on the synthesis conditions, MFI zeolites may contain a heterogeneous distribution of acid sites positioned in isolated configurations or in close proximity to another acid site, of which proximate acid sites can be quantified by cobalt titration techniques. These acid sites may be located within straight or sinusoidal channels (around 0.55 nm void size diameter) or within the larger intersections (around 0.65 nm in void size diameter). Void size is defined as the average cross-sectional diameter of the channels or their intersections as specified in the IZA database. For a given Si/Al ratio, the fraction of proximate Al species may be controlled by varying the organic and inorganic structure directing agent used during MFI zeolite synthesis. The distribution of acid sites within zeolites in different pore locations or at different relative proximity has been identified as a parameter that affects various acid-catalyzed reactions.

On the other hand, MFI prepared according to the disclosure is shown to produce para-xylene with minor amounts of the other xylenes and negligible amounts of consecutive methylated product observed. This reactivity pattern would translate to low propensity for side product formation and thus higher methanol utilization.

This reactivity pattern under the TM diagnostic test reaction condition further characterizes MFI samples with altered Al distributions, with highly dispersed framework aluminum or a lack of proximate framework aluminum. Comparative examples synthesized by the conventional OSDA (TPA) have appreciable fractions of framework aluminum in close proximity to each other as shown in Table 1 below. Proximity of framework aluminum may be further characterized by measurement of the amount of cobalt ion exchanged onto MFI zeolite during the “cobalt titration technique” as shown in Tables 1 and 2. Combined, the TM diagnostic test and the cobalt titration technique may be the best available technique for characterization of framework aluminum proximity.

MFI zeolites of the instant disclosure possess fewer than 18% proximate aluminum framework sites by the cobalt titration technique whose value is shown as 2×Co²⁺/Al in Table 1. Values in this test may range from 0 to 1, thus from 0% to 100%. They may possess less than 15% or less than 10% proximate aluminum framework sites by the cobalt titration technique.

The methanol utilization via using MFI zeolite intrinsically more selective for para-xylene production, would be higher, since it requires less catalyst selectivation to attain a given level para-xylene selectivity, thus less methanol utilization trade-off. The attributes contributing to more para-xylene selectivity performance is homogeneous and make it more reproducible than conventional selectivation means. This material enables toluene methylation process to operate at lower temperatures, shorter contact time due to less selectivation required to achieve comparable to higher para-xylene productivity, and thus higher methanol utilization.

An intrinsically more selective MFI zeolite of high para-xylene and lower light olefin and paraffin production would enable higher para-xylene production throughput and lower production cost due to its significantly higher methanol utilization (or methanol consumption) for increasing methyl to phenyl ratios coming off aromatics complex.

In accordance with an exemplary embodiment, a process is provided for producing para-xylene comprising reacting oxygenates with an aromatic feedstock comprising toluene and/or benzene in a methylation zone operating under alkylation conditions comprising a maximum temperature of about 400° C. to about 675° C. and a pressure of about 10 kPa to 5000 kPa in the presence of a catalyst comprising an improved MFI zeolite to provide a product stream comprising para-xylene.

In accordance with another exemplary embodiment, a process is provided for producing para-xylene comprising reacting a toluene stream and a methanol stream in a toluene methylation zone operating under toluene methylation conditions comprising a maximum temperature of about 400° C. to about 675° C. and a pressure of about 10 kPa to 5,000 kPa in the presence of a catalyst composition comprising an improved MFI zeolite to produce a product stream comprising para-xylene.

In accordance with yet another exemplary embodiment, a process is provided for producing para-xylene comprising reacting a toluene stream and a methanol stream in a toluene methylation zone operating under toluene methylation conditions comprising a maximum temperature of about 400° C. to about 675° C., a pressure of about 10 kPa to 5,000 kPa, a weight hourly space velocity of from 0.5 to 20 hr⁻¹ and a toluene to methanol molar ratio of from about 1:1 to 6:1, in the presence of a catalyst composition comprising an improved MFI zeolite to produce to produce a product stream comprising para-xylene.

The feed stream to the present process generally comprises alkylaromatic hydrocarbons of the general formula C₆H⁽⁶⁻ⁿ⁾Rₙ, where n is an integer from 0 to 5 and each R may be CH₃, C₂H₅, C₃H₇, or C₄H₉, in any combination. The aromatics-rich feed stream to the process of the present disclosure may be derived from a variety of sources, including without limitation conventional catalytic reforming, zeolitic reforming converting C₆-C₇ non-aromatics from light naphtha or aromatic extraction raffinates to benzene and toluene, steam pyrolysis of naphtha, distillates or other hydrocarbons to yield light olefins and aromatics-rich byproducts (including gasoline-range material often referred to as “pygas”), and catalytic or thermal cracking of distillates and heavy oils to yield products in the gasoline range. Products from pyrolysis or other cracking operations generally will be hydrotreated according to processes well known in the industry before being charged to the complex in order to remove sulfur, olefins and other compounds which would affect product quality and/or damage catalysts and downstream process. Light cycle oil from catalytic cracking also may be beneficially hydrotreated and/or hydrocracked according to known technology to yield products in the gasoline range; the hydrotreating preferably also applies to catalytic reforming to yield the aromatics-rich feed stream. The feed stream may predominantly comprise toluene.

In an aspect, the aromatic feedstock comprises toluene. In another aspect, the aromatic feedstock may include benzene. In an embodiment, the aromatic feedstock may include both benzene and toluene. The process condition for formation of para-xylene may include a maximum temperature of from about of about 400° C. to about 675° C., preferably from about 450° C. to about 650° C. and more preferably from about 500° C. to about 625° C. In accordance with various embodiments, the maximum temperature may refer to the maximum temperature of the catalyst bed and may be interchangeably referred to as the maximum bed temperature. Further, the process condition may include a pressure of from about 10 kPa to 5,000 kPa, preferably from about 100 kPa to 2000 kPa and more preferably from about 300 kPa to about 1000 kPa. The process conditions may further include a weight hourly space velocity (WHSV) of from 0.1 to 25 hr⁻¹, preferably from about 0.5 to 15 hr⁻¹ and more preferably from about 2 to 12 hr⁻¹. Also, the alkylation conditions may include an aromatic feedstock to oxygenate molar ratio of from about 0.5:1 to 10:1, preferably from about 1:1 to 6:1 and more preferably from about 1.5:1 to 4:1. In an embodiment, the conditions may comprise a maximum temperature of less than about 650° C., a pressure of about 100 kPa to 1,000 kPa, and a toluene to methanol molar ratio of from about 1:2 to 6:1. The oxygenates may be selected from the group consisting of methanol, dimethylether, dimethyl carbonate, and mixtures thereof.

Diluents may also comprise the feed stream. Diluents may comprise H₂, H₂O, and combinations thereof. The molar ratio of diluent to aromatic feedstock and oxygenate feedstock may range from 0.1 to 3.0, preferably from 0.1 to 2.0 and most preferably from 0.2 to 1.5. In an aspect, the molar ratio may be described as H₂O/(toluene+methanol) and may range from 0.1 to 3.0.

In this disclosure we tailor the morphology of zeolite MFI independent of the distribution of framework aluminum within the 12 T-sites of the MFI structure. Distribution of the framework aluminum may be probed through the use of the cobalt titration technique and/or NH₃ TPD. The improved MFI zeolite may comprise a heteroatom Q selected from the group consisting of boron, gallium, indium and iron, and mixtures thereof. Incorporation of heteroatom such as boron in addition to framework aluminum may reduce the effective mass transport path across the crystallite as shown in Tables 1 and 2 below. The heteroatom Q may be boron and the ratio of SUB may range from about 20 to about 50. Via incorporating boron and specific OSDA including ethylenediamine (EDA) and 1,4 diazobicyclo[2.2.2]octane (DABCO) with SiO₂/Al₂O₃ ratios ranging from 40 to 1000 in MFI syntheses, improved methanol utilization can be achieved via highly dispersed framework aluminum for reduced MTH side reactions and via improved mass transport properties for enhanced toluene methylation.

The improved MFI zeolite of the disclosure may be formulated into the catalyst through combination with binders. The improved MFI zeolite may comprise between about 25% and about 95% of the catalyst by weight.

### EXAMPLES

### Example 1

Zeolite MFI used in the subjected disclosure for selective methylation of aromatics such as toluene to para-xylene was synthesized via the use of Structural Directing Agent (SDA) comprising ethylene diamine (EDA) and/or 1,4 diazobicyclo[2.2.2]octane (DABCO) using the procedures described. MFI syntheses could contain heteroatom such as boron (designated as B-Al-MFI as opposed to Al-MFI) to reduce zeolite size to attain favorable mass transport properties. Al-MFI and B-Al-MFI synthesized using EDA have Si/Al ratios ranging from about 50 to about 1000. Si/Al is related to SiO₂/Al₂O₃ by a factor of 2. That is, a zeolite with Si/Al=50 possesses a SiO₂/Al₂O₃ of 100. They are characterized by highly dispersed distributions of framework aluminum measured by ion exchanges of zeolite with cobalt ions (the “cobalt titration technique”) as shown in Tables 1 and 2. The experimental procedure for the cobalt titration technique is described in C. T. Nimlos, “Theoretical and Experimental Assessment of Aluminum Proximity in MFI Zeolite and its Alteration by Organic and Inorganic Structural Directing Agents” in Chem. Mater. 2020, 32 (21), 9277-9298. MFI zeolites of the instant disclosure possess fewer than 18% proximate aluminum framework sites by the cobalt titration technique whose value is shown as 2×Co²⁺/Al in Table 1. They may possess less than 15% or less than 10% proximate aluminum framework sites by the cobalt titration technique.

### Example 1.1

Zeolite MFI was synthesized using a combination of boron and aluminum as the heteroatoms and using a combination of EDA and TPA as the SDAs according to the procedure of Y. G. Hur, “Influence of tetrapropyl ammonium and Ethylenediamine Structural Directing Agents on Framework Aluminum Distribution” in Ind. Eng. Chem. Res. 2019, 58 (27), 11849-11860 and J. T. Miller, “Increased Oligomer Selectivity in Olefin Oligomerization by Incorporation of Boron” in WO2019028035A2.

### Example 1.2

Zeolite MFI was synthesized using aluminum as the heteroatom using DABCO as the SDA according to the procedure of C. T. Nimlos previously mentioned.

### Example 1.3

Zeolite MFI was synthesized using aluminum as the heteroatom using EDA and TPA as the SDA according to the procedure of Y. G. Hur previously mentioned and J. T. Miller and co-inventors on “Increased Oligomer Selectivity in Olefin Oligomerization by Incorporation of Boron” in WO2019028035A2.

### Example 1.4

Zeolite MFI was synthesized using aluminum as the heteroatom using EDA and TPA as the SDAs according to the procedure of Y. G. Hur previously mentioned and J. T. Miller and co-inventors on “Increased Oligomer Selectivity in Olefin Oligomerization by Incorporation of Boron” in WO2019028035A2.

### Example 1.5

Zeolite MFI was synthesized using a combination of boron and aluminum as the heteroatoms and using a combination of EDA and TPA as the SDAs according to the procedure of Y. G. Hur previously mentioned and J. T. Miller and co-inventors on “Increased Oligomer Selectivity in Olefin Oligomerization by Incorporation of Boron” in WO2019028035A2.

### Comparative Example 2

For comparative purposes to illustrate the distinct reactivity patterns of Zeolite MFI of the subject disclosure, a series of MFI with high fraction of populations of proximate framework aluminum sites were synthesized at similar Si/Al ratios using tetrapropyl ammonium (TPA) OSDAs in Comparative Examples. Also included here are commercial MFI zeolite samples. The characteristics of the samples are summarized is Tables 1 and 2.

### Example 2.1

Zeolite MFI was purchased from Zeolyst as CBV8014 at Si/Al=43.

### Example 2.2

Zeolite MFI was synthesized using a combination of boron and aluminum as the heteroatom using TPA as the SDA according to the procedure of Y. G. Hur previously mentioned and WO2019028035A2.

### Example 2.3

Zeolite MFI was synthesized using aluminum as the heteroatom using TPA as the SDA according to the procedure of C. T. Nimlos previously mentioned.

### Example 2.4

Zeolite MFI was synthesized using aluminum as the heteroatom using TPA and Na as the SDAs according to the procedure of C. T. Nimlos previously mentioned.

TABLE 1

| Example | 1.1 | 1.2 | 1.3 | 1.4 | 1.5 |
|---|---|---|---|---|---|
| Synthesis SDAs | TPA⁺ + | DABCO + | TPA⁺ + | TPA⁺ + | TPA⁺ + |
| | EDA | Na⁺ + | EDA | EDA | EDA |
| | | CH₃NH₂ | | | |
| Framework | B-Al-MFI | Al-MFI | Al-MFI | Al-MFI | B-Al-MFI |
| Si/Al | 53 | 44 | 58 | 49 | ~50 |
| H⁺/Al | 1.02 | 0.87 | 0.92 | 0.6 | — |
| Al/u.c. | 1.72 | 2.13 | 1.63 | 1.89 | ~2 |
| H⁺/u.c. | 1.75 | 1.85 | 1.50 | 1.91 | |
| Si/B | 26 | B-free | B-free | B-free | ~25 |
| 2 × Co²⁺/Al | 0.01 | 0.06 | 0.01 | 0.01 | — |
| Avg. crystallite | 1.3 ± 0.3 μm | 5 × 5 × 10 μm | 8 μm | — | — |
| size (SEM) | | | | | |

TABLE 2

| Example | 2.1 | 2.2 | 2.3 | 2.4 |
|---|---|---|---|---|
| Synthesis SDAs | Unknown | TPA⁺ | TPA⁺ | TPA⁺ + Na |
| Framework | Al-MFI | B-Al-MFI | Al-MFI | Al-MFI |
| Si/Al | 43 | 59 | 50 | 55 |
| H⁺/Al | 0.85 | 0.84 | 1.01 | 0.97 |
| Al/u.c. | 2.18 | 1.57 | 1.89 | 1.73 |
| H⁺/u.c. | 1.85 | 1.31 | 1.91 | 1.68 |
| Si/B | B-free | 48 | B-free | B-free |
| 2 × Co²⁺/Al | 0.46 | 0.20 | 0.24 | 0.44 |
| Avg. crystallite | 0.3 μm | ~0.6 μm | 0.5 μm | 4 μm |
| size(SEM) | | | | |

### Example 2 Toluene Methylation

The zeolite of the subject disclosure may further be characterized by a catalytically diagnostic test performed at 130° C. and dimethylether (DME) to toluene molar ratio of 16 with active sites being mostly covered by oxygenates. This test is the TM diagnostic test. The diagnostic test conditions limit toluene methylation at less than 5% conversions, is designed to probe and characterize active sites for the toluene methylation under kinetically controlled reaction regimes with results summarized in Table 3 below.

Specifically, toluene methylation experiments were conducted in a tubular packed-bed reactor (quartz, 7 mm ID) at 403 K. Fresh zeolite samples (0.010-0.060 g; NH₄⁺-form) were pelleted, crushed, and sieved to retain aggregates between 180 and 250 μm in diameter. The sieved samples were diluted with acid-purified quartz sand (180-250 μm) to maintain a constant 1 g of solid material which was supported between two plugs of quartz wool. The bed temperature was measured using a K-type thermocouple in contact with the side of the quartz tube at the level of the bed and maintained at desired temperature using a three-zone furnace (Applied Test Systems) and Watlow controllers (EZ-ZONE). For higher conversion studies, a higher mass (1.8 g) of MFI sample without silica diluent was evaluated at DME to toluene molar ratios of 6 and at temperatures of 130° C.

Prior to catalytic runs, catalysts were pre-treated (5 K/min to 773 K) in 5% O₂/He flow (UHP, Indiana Oxygen, 100 cm³/min), After a 4 h hold, the catalysts were cooled down (5 K/min) to reaction temperature and flushed with He for at least 1 h before reactants were introduced. Liquid toluene (Sigma Aldrich, HPLC grade, >99.99%) was vaporized at a heated tee (473 K) into a mixed stream of He (UHP, Indiana Oxygen) and DME (Matheson, CP, >99.5%) with the aid of a syringe pump (KD Scientific Legato 100). For toluene methylation with methanol, the methanol was premixed with toluene in desired molar ratios and fed into the same tee. All heated lines upstream of reactor were kept >400 K while heated lines from reactor outlet to GC were maintained >440 K to limit condensation, Methane (0-5 cm³/min; 25% CH₄/Ar; Indiana Oxygen) was co-fed with the reactants and used as internal standard. Total flow rate of stream was maintained between 50-100 cm³/min. Reactant and product concentrations were measured (25-30 min sampling intervals) by online gas chromatography (Agilent 7890B) using DR-Wax column (30 m×320 μm×0.5 μm) and flame ionization detector, GC peak areas were quantified using calibration curves developed from feeding known quantities of standards to the GC.

Prior to reaction, the feed stream composition was stabilized and verified from bypass injections. The reactions were run at 4 kPa toluene and 66 kPa DME (or 4 kPa methanol) and fixed reaction conditions for 6-14 h while initial conversions were kept below 0.4%. No products were observed during blank reactor tests with quartz wool and SiO₂ at 473 K. Xylenes site time yield (STY) are calculated from the reactor outlet molar flow rates of xylenes normalized by initial proton counts (obtained using NH₃ TPD) at start of the reaction. TPD conditions and procedures are described in detail in C. T. Nimlos previously mentioned. Xylenes selectivity are calculated from the individual xylenes STY normalized by total xylenes STY. Toluene conversions are calculated on a product mole basis. Initial rates, xylene selectivity and conversion are reported at 0.2-0.5 h time on stream. In one case, an MFI catalyst had its external acidity poisoned using 2,6-di-tertbutylpyridine (DTBP) at 0.006-0.022 kPa partial pressure that was co-fed with toluene.

TABLE 3

| Example | 1.1 | 1.2 | 1.3 | 1.4 | 1.5 | 2.1 | 2.2 | 2.3 | 2.4 |
|---|---|---|---|---|---|---|---|---|---|
| Initial | 0.079 | 0.152 | 0.079 | 0.09 | 0.07 | 0.559 | 0.476 | 0.344 | 0.421 |
| Xylene STY | | | | | | | | | |
| (mmol | | | | | | | | | |
| xylene/H⁺/s) | | | | | | | | | |
| Xylene STY | 0.00441 | 0.00596 | 0.00618 | — | — | 0.152 | 0.103 | 0.0806 | 0.0728 |
| at 9 h TOS | | | | | | (5 h TOS) | | | |
| (mmol | | | | | | | | | |
| xylene/H⁺/s) | | | | | | | | | |
| Initial | 85/2/13 | 74/2/24 | 74/2/24 | 71/1/28 | 71/5/25 | 28/8/64 | 26/7/67 | 27/8/65 | 30/5/65 |
| Xylene | | | | | | | | | |
| Selectivity | | | | | | | | | |
| (pX/mX/oX) | | | | | | | | | |
| Xylene | N/D | N/D | N/D | N/D | N/D | 27/17/56 | 27/16/57 | 25/17/58 | 26/17/57 |
| Selectivity at | | | | | | (5 h TOS) | | | |
| 9 h TOS | | | | | | | | | |
| (pX/mX/oX) | | | | | | | | | |

The catalytic performance of the disclosed MFI zeolites in the TM diagnostic test is characterized by having initial activities defined as total xylene STY of 5 to 10 times lower than comparative example MFI zeolites synthesized by conventional means and/or SDA such as tetrapropylammonium. STY is specified in unit of moles product/(moles H^{+)-second and originally defined by Boudart in Chem. Rev.} 1995, 95, 661-666. Under the kinetically controlled reaction region, the zeolite particle size controlled via the incorporation of boron in syntheses does not play a role in activity or preference in formation of specific xylene isomer.

The catalytic performance of the disclosed MFI zeolites in the TM diagnostic test is further characterized by having initial para-xylene content of greater than 70% within the total xylenes, double that observed in MFI zeolites synthesized by conventional SDA. The catalytic performance of the disclosed MFI zeolites, synthesized by EDA or DABCO SDA, is further characterized by having steady state para-xylene to total xylenes of greater than about 30% or greater than 35%. Comparative example MFI zeolites synthesized by conventional SDA exhibit para-xylene selectivity of about 20% to about 27% of the total xylenes as shown in the attached summary. Xylene selectivity values are not determined (N/D) for runs where STY is less than about 0.03 as insignificant quantities of xylenes are produced to reliably determine the specific xylenes fractions.

The low toluene methylation activity and high para-xylene selectivity are characteristic of active sites configured for more shape selective isomer, i.e. para-xylene in toluene methylation reaction and also less consecutive methylated products, i.e. polymethylated benzene (PMB). As previously pointed out, the characteristics of the active sites of the instant disclosure in MFI zeolites characterized by having low populations of proximate framework aluminum sites are less prone to facilitate MTH reactions, thus intrinsically more selective to aromatics methylation to xylene and thus enhancing methanol utilization.

At 130° C. and higher toluene conversions up to 6%, the sample described in example 2.1, ortho-xylene remains the major product with selectivity of 64% to 72% at all times on stream while para-xylene (22 to 24%) and meta-xylene (6% to 12%) remain minor products at all times on stream. Xylenes compose >95% of total aromatic products.

At 130° C. the sample described in example 1.4 is evaluated in presence and absence of a poison (DTBP) that selectively suppresses external acidity. The initial xylenes selectivity remains similar and dominant in para-xylene (69-71%) but the xylenes selectivity at longer time on streams (9 h) has a higher para-xylene selectivity (45%) compared to that (34%) in the absence of co-fed DTBP.

TABLE 4

| Example | 1.4 | 1.4 | 2.1 | 2.1 |
|---|---|---|---|---|
| Toluene Conversion (%) | 0.5 | 0.5 | 0.3 | 5.7 |
| DTBP Pressure (kPa) | 0 | 0.022 | 0 | 0 |
| Initial Xylene Selectivity | 68/1/31 | 71/1/28 | 28/8/64 | 22/6/72 |
| (pX/mX/oX) | | | | |
| Xylene Selectivity at 9 h | 34/14/52 | 45/10/45 | 27/17/56 | 24/12/64 |
| TOS (pX/mX/oX) | | | (5 h TOS) | |

### Examples 4-6

To further minimize the fractions of proximate framework aluminum, EDA-MFI syntheses were conducted at increasing SiO₂/Al₂O₃ ratios, while boron is incorporated into the synthesis to maintain particle sizes of favorable mass transport properties as illustrated below in Examples 4 through 6 accompanied with corresponding Comparative Examples prepared at comparable SiO₂/Al₂O₃ ratios. The properties of the resulting Zeolite MFI are summarized in Table 3.

### Example 4

An alumina-boro-silicate solution was prepared by first mixing 11.19 g of aluminum nitrate nonahydrate, 70.09 g boric acid, 53.77 g of ethylenediamine, 30.33 g of TPAOH (40% solution) and 791.45 g of water, while stirring vigorously. After thorough mixing, 443.18 g Ludox HS-40 (SiO₂, mass-40%). The reaction mixture was homogenized for 20 minutes with a high-speed mechanical stirrer, transferred to a 2-L Parr Hastelloy stir autoclave. The mixture was crystallized at 175° C. with stirring at 300 RPM for 88 hours. The solid product was recovered by filtration, washed with de-ionized water, and dried at 100° C. The product was identified as MFI by XRD. Chemical analysis gave a product composition of Si/Al=111, Si/B=21.36. The sample was calcined at 580° C. x 6 hrs. and the BET surface area was 287 m²/g with a micropore volume of 0.143 cc/g and a total pore volume of 0.164 cc/g.

### Comparative Example 4A

An alumina-boro-silicate solution was prepared by first mixing 7.53 g of aluminum nitrate nonahydrate, 47.18 g boric acid, 306.21 g of TPAOH (40% solution) and 740.74 g of water, while stirring vigorously. After thorough mixing, 298.33 g Ludox HS-40 (SiO₂, mass-40%). The reaction mixture was homogenized for 20 minutes with a high-speed mechanical stirrer, transferred to a 2-L Parr Hastelloy stir autoclave. The mixture was crystallized at 175° C. with stirring at 300 RPM for 88 hours. The solid product was recovered by filtration, washed with de-ionized water, and dried at 100° C. The product was identified as MFI by XRD. Chemical analysis gave a product composition of Si/Al=88, Si/B=35, The sample was calcined at 580° C. x 6 hrs. and the BET surface area was 344 m²/g with a micropore volume of 0.171 cc/g and a total pore volume of 0.190 cc/g.

### Comparative Example 4B

Zeolite silicalite of target SiO₂/Al₂O₃ ratio of 95 was synthesized using tetrapropylammonium (TPA) as an OSDA. The resulting zeolite after calcination to remove the organic template has Si/Al ratio of 100 and a BET surface area of 358 m²/gm with a micropore volume of 0.184 cc/g and a total pore volume of 0.208 cc/g.

### Example 5

An alumina-boro-silicate solution was prepared by first mixing 5.61 g of aluminum nitrate nonahydrate, 70.31 g boric acid, 53.94 g of ethylenediamine, 30.42 g of TPAOH (40% solution) and 795.15 g of water, while stirring vigorously. After thorough mixing, 444.57 g Ludox HS-40 (SiO₂, mass-40%). The reaction mixture was homogenized for 20 minutes with a high-speed mechanical stirrer, transferred to a 2-L Parr Hastelloy stir autoclave. The mixture was crystallized at 175° C. with stirring at 300 RPM for 88 hours. The solid product was recovered by filtration, washed with de-ionized water, and dried at 100° C. The product was identified as MFI by XRD. Chemical analysis gave a product composition of Si/Al=210.5, Si/B=19.27. The sample was calcined at 580° C. x 6 hrs. and the BET surface area was 287 m²/g with a micropore volume of 0.145 cc/g and a total pore volume of 0.157 cc/g.

### Example 6

An alumina-boro-silicate solution was prepared by first mixing 3.74 g of aluminum nitrate nonahydrate, 70.39 g boric acid, 54.0 g of ethylenediamine, 30.45 g of TPAOH (40% solution) and 796.39 g of water, while stirring vigorously. After thorough mixing, 445.03 g Ludox HS-40 (SiO₂, mass-40%). The reaction mixture was homogenized for 20 minutes with a high-speed mechanical stirrer, transferred to a 2-L Parr Hastelloy stir autoclave. The mixture was crystallized at 175° C. with stirring at 300 RPM for 88 hours. The solid product was recovered by filtration, washed with de-ionized water, and dried at 100° C. The product was identified as MFI by XRD. Chemical analysis gave a product composition of Si/Al=295.3, Si/B=18.85. The sample was calcined at 580° C. x 6 hrs. and the BET surface area was 285 m²/g with a micropore volume of 0.144 cc/g and a total pore volume of 0.154 cc/g.

### Comparative Example 6A

An alumina-boro-silicate solution was prepared by first mixing 3.02 g of aluminum nitrate nonahydrate, 47.3 g boric acid, 306.99 g of TPAOH (40% solution) and 743.6 g of water, while stirring vigorously. After thorough mixing, 299.09 g Ludox HS-40 (SiO₂, mass-40%). The reaction mixture was homogenized for 20 minutes with a high-speed mechanical stirrer, transferred to a 2-L Parr Hastelloy stir autoclave. The mixture was crystallized at 175° C. with stirring at 300 RPM for 88 hours. The solid product was recovered by filtration, washed with de-ionized water, and dried at 100° C. The product was identified as MFI by XRD. Chemical analysis gave a product composition of Si/Al=240, Si/B=35. The sample was calcined at 580° C.×6 hrs. and the BET surface area was 342 m²/g with a micropore volume of 0.173 cc/g and a total pore volume of 0.184 cc/g.

### Comparative Example 6B

Zeolite silicalite of target SiO₂/Al₂O₃ ratio of 500 was synthesized using tetrapropylammonium (TPA) as an OSDA. The resulting zeolite after calcination to remove the organic template has Si/Al ratio of 236 and a BET surface area of 353 m²/gm with a micropore volume of 0.170 cc/g and a total pore volume of 0.209 cc/g.

TABLE 3

| | SiO₂/Al₂O₃ | | | BET | | Total |
|---|---|---|---|---|---|---|
| Example | target | SiO₂/Al₂O₃ | Si/B₂O₃ | SA | uPV | PV |
| 4 | 221 | 221 | 42.3 | 287 | 0.143 | 0.164 |
| 4A | 200 | 175 | 70 | 344 | 0.171 | 0.190 |
| 4B | 190 | 199 | boron | 358 | 0.184 | 0.208 |
| | | | free | | | |
| 5 | 400 | 421 | 38.55 | 287 | 0.145 | 0.157 |
| 6 | 591 | 590 | 37.7 | 285 | 0.144 | 0.154 |
| 6A | 500 | 480 | 70 | 342 | 0.173 | 0.184 |
| 6B | 500 | 472 | boron | 353 | 0.170 | 0.209 |
| | | | free | | | |

### Example 7 (Catalyst Preparation)

The aforementioned Zeolite MFI's representing Examples and Comparative Examples can be formulated into the form of either extrudate or spray dry particle containing 10 to 75% zeolite and 25 to 90% binder. Binders are silica, alumina and silica alumina. Clay binder is incorporated into the formation at a content of 20 to 60% to densify and strengthen the spray dry particles. Preferably, alkali earth oxides such as MgO and/or phosphate are incorporated into the catalyst formulation to entail para-xylene selectivity in toluene methylation process. Also, preferably the catalyst is subject to steam treatments with the severity ranging from 500 to 1100° C. at greater than 80% steam contents over a period of 30 minutes to 48 hours.

### Example 8 (Toluene Methylation Tests)

MFI zeolites of the subject disclosure can be deployed under the process conditions with toluene to methanol molar ratios ranging from 1.5 to 6.0, a temperature range from 400° C. to 675° C., WHSV range from 2 to 20 hr⁻¹ and a pressure range from 100 to 1000 psig. Optionally H₂, H₂O, or H₂ and H₂O is co-fed with toluene and methanol to improve para-xylene selectivity and methanol utilization. Methanol utilization is expected to be greater than 50% or greater than 60% or greater than 70% or greater than 80%. The molar ratios of H₂/(toluene+methanol) and H₂O/(toluene+methanol) can range from 0.1 to 3.0, preferably from 0.1 to 2.0 and most preferably from 0.2 to 1.5. The catalysts can be deployed in a fixed bed process with occasional regeneration or a fluidized or riser bed with frequent regeneration.

### Specific Embodiments

While the following is described in conjunction with specific embodiments, it will be understood that this description is intended to illustrate and not limit the scope of the preceding description and the appended claims.

A first embodiment of the invention is a process for the production of para-xylene comprising contacting a feed stream comprising an oxygenate feedstock and an aromatic feedstock comprising toluene with a catalyst, converting the feed stream to a product at reaction conditions, and recovering a product comprising para-xylene, wherein the catalyst comprises an improved MFI zeolite comprising in the calcined and ion-exchanged form a SiO₂/Al₂O₃ ratio of from about 50 to about 600 and having a low population of proximate framework aluminum sites characterized by an initial xylene selectivity of greater than 70% in a TM diagnostic test. An embodiment of the invention is one, any or all of prior embodiments in this paragraph up through the first embodiment in this paragraph wherein the product comprises one or more xylenes. An embodiment of the invention is one, any or all of prior embodiments in this paragraph up through the first embodiment in this paragraph wherein the SiO₂/Al₂O₃ ratio is from about 80 to about 550. An embodiment of the invention is one, any or all of prior embodiments in this paragraph up through the first embodiment in this paragraph wherein the low population of proximal framework aluminum sites is further characterized by a value of less than 18% by the cobalt titration technique. An embodiment of the invention is one, any or all of prior embodiments in this paragraph up through the first embodiment in this paragraph wherein the improved MFI zeolite further comprises a heteroatom Q selected from the group consisting of boron, gallium, indium and iron, and mixtures thereof. An embodiment of the invention is one, any or all of prior embodiments in this paragraph up through the first embodiment in this paragraph wherein the molar ratio of Si/Q is between about 2 and about 100. An embodiment of the invention is one, any or all of prior embodiments in this paragraph up through the first embodiment in this paragraph wherein the improved MFI zeolite comprises between about 10% and about 75% of the catalyst by weight. An embodiment of the invention is one, any or all of prior embodiments in this paragraph up through the first embodiment in this paragraph wherein the catalyst further comprises greater than 0 wt % and less than 5 wt % phosphorus. An embodiment of the invention is one, any or all of prior embodiments in this paragraph up through the first embodiment in this paragraph wherein the catalyst further comprises greater than 0 wt % and less than 1 wt % calcium, magnesium, or mixtures thereof. An embodiment of the invention is one, any or all of prior embodiments in this paragraph up through the first embodiment in this paragraph wherein the oxygenate is selected from methanol, dimethyl ether, dimethyl carbonate or a mixture of thereof. An embodiment of the invention is one, any or all of prior embodiments in this paragraph up through the first embodiment in this paragraph wherein the reaction conditions comprise a maximum temperature of from about of about 400° C. to about 675° C. An embodiment of the invention is one, any or all of prior embodiments in this paragraph up through the first embodiment in this paragraph wherein the reaction conditions comprise a pressure of from about 10 kPa to 5,000 kPa. An embodiment of the invention is one, any or all of prior embodiments in this paragraph up through the first embodiment in this paragraph wherein the reaction conditions comprise an aromatic feedstock to oxygenate molar ratio of from about 0.5:1 to 10:1. An embodiment of the invention is one, any or all of prior embodiments in this paragraph up through the first embodiment in this paragraph wherein the reaction conditions comprise a weight hourly space velocity (WHSV) of from 0.1 to 20 hr⁻¹. An embodiment of the invention is one, any or all of prior embodiments in this paragraph up through the first embodiment in this paragraph wherein the feed stream comprises a diluent selected from the group consisting of H₂, H₂O, H₂, and combinations thereof. An embodiment of the invention is one, any or all of prior embodiments in this paragraph up through the first embodiment in this paragraph wherein the molar ratio of diluent to aromatic feedstock and oxygenate feedstock may range from 0.1 to 3.0.

A second embodiment of the invention is a process for the production of para-xylene comprising contacting a feed stream comprising an oxygenate feedstock and an aromatic feedstock comprising toluene with a catalyst, converting the feed stream to a product at reaction conditions, and recovering a product comprising para-xylene, wherein the catalyst comprises an improved MFI zeolite comprising in the calcined and ion-exchanged form a SiO₂/Al₂O₃ ratio of from about 100 to about 500, a heteroatom Q selected from the group consisting of boron, gallium, indium and iron, and mixtures thereof wherein the molar ratio of Si/Q is between about 2 and about 100 and having a low population of proximate framework aluminum sites characterized by an initial xylene selectivity of greater than 70% in a TM diagnostic test. An embodiment of the invention is one, any or all of prior embodiments in this paragraph up through the second embodiment in this paragraph wherein the reaction conditions comprise an aromatic feedstock to oxygenate molar ratio of from about 0.5:1 to 10:1, a weight hourly space velocity (WHSV) of from 0.1 to 20 hr⁻¹, and a pressure of from about 10 kPa to 5,000 kPa.

A third embodiment of the invention is a process for the production of para-xylene comprising contacting a feed stream comprising methanol and toluene with a catalyst, converting the feed stream to a product at reaction conditions, and recovering a product comprising para-xylene, wherein between about 10% and about 75% by weight of the catalyst comprises an improved MFI zeolite comprising in the calcined and ion-exchanged form a SiO₂/Al₂O₃ ratio of from about 100 to about 500, a heteroatom Q selected from the group consisting of boron, gallium, indium and iron, and mixtures thereof wherein the molar ratio of Si/Q is between about 2 and about 100 and having a low population of proximate framework aluminum sites characterized by an initial xylene selectivity of greater than 70% in a TM diagnostic test. An embodiment of the invention is one, any or all of prior embodiments in this paragraph up through the third embodiment in this paragraph wherein the reaction conditions comprise an aromatic feedstock to oxygenate molar ratio of from about 0.5:1 to 10:1, a weight hourly space velocity (WHSV) of from 0.1 to 20 hr⁻¹, and a pressure of from about 10 kPa to 5,000 kPa, An embodiment of the invention is one, any or all of prior embodiments in this paragraph up through the third embodiment in this paragraph wherein the product possesses a methyl to phenyl ratio of greater than about 1.75 and less than about 2.0. An embodiment of the invention is one, any or all of prior embodiments in this paragraph up through the third embodiment in this paragraph wherein a methanol utilization is greater than 60%.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for the production of para-xylene comprising:
contacting a feed stream comprising an oxygenate feedstock and an aromatic feedstock comprising toluene with a catalyst,
converting the feed stream to a product at reaction conditions, and
recovering a product comprising para-xylene,
wherein the catalyst comprises an improved MFI zeolite comprising in the calcined and ion-exchanged form a SiO₂/Al₂O₃ ratio of from about 50 to about 600 and having a low population of proximate framework aluminum sites characterized by an initial xylene selectivity of greater than 70% in a TM diagnostic test.

2. The process of claim 1 wherein the product comprises one or more xylenes.

3. The process of claim 1 wherein the SiO2/Al2O3 ratio is from about 80 to about 550.

4. The process of claim 1 wherein the low population of framework aluminum sites is further characterized by a value of less than 18% by the cobalt titration technique.

5. The process of claim 1 wherein the improved MFI zeolite further comprises a heteroatom Q selected from the group consisting of boron, gallium, indium and iron, and mixtures thereof.

6. The process of claim 5 wherein the molar ratio of Si/Q is between about 20 and about 100.

7. The process of claim 1 wherein the improved MFI zeolite comprises between about 10% and about 75% of the catalyst by weight.

8. The process of claim 1 wherein the catalyst further comprises greater than 0 wt % and less than 5 wt % phosphorus.

9. The process of claim 8 wherein the catalyst further comprises greater than 0 wt % and less than 1 wt % calcium, magnesium, or mixtures thereof.

10. The process of claim 1 wherein the oxygenate is selected from methanol, dimethyl ether, dimethyl carbonate or a mixture of thereof.

11. The process of claim 1 wherein the reaction conditions comprise a maximum temperature of from about of about 400° C. to about 675° C.

12. The process of claim 1 wherein the reaction conditions comprise a pressure of from about 10 kPa to 5,000 kPa.

13. The process of claim 1 wherein the reaction conditions comprise an aromatic feedstock to oxygenate molar ratio of from about 0.5:1 to 10:1.

14. The process of claim 1 wherein the reaction conditions comprise a weight hourly space velocity (WHSV) of from 0.1 to 20 hr⁻¹.

15. The process of claim 1 wherein the feed stream comprises a diluent selected from the group consisting of H₂, H₂O, H₂, and combinations thereof.

16. The process of claim 15 wherein the molar ratio of diluent to aromatic feedstock and oxygenate feedstock may range from 0.1 to 3.0.

17. A process for the production of para-xylene comprising:
contacting a feed stream comprising an oxygenate feedstock and an aromatic feedstock comprising toluene with a catalyst,
converting the feed stream to a product at reaction conditions, and
recovering a product comprising para-xylene,
wherein the catalyst comprises an improved MFI zeolite comprising in the calcined and ion-exchanged form a SiO₂/Al₂O₃ ratio of from about 100 to about 500, a heteroatom Q selected from the group consisting of boron, gallium, indium and iron, and mixtures thereof wherein the molar ratio of Si/Q is between about 2 and about 100 and having a low population of proximate framework aluminum sites characterized by an initial xylene selectivity of greater than 70% in a TM diagnostic test.

18. The process of claim 17 wherein the reaction conditions comprise an aromatic feedstock to oxygenate molar ratio of from about 0.5:1 to 10:1, a weight hourly space velocity (WHSV) of from 0.1 to 20 hr⁻¹, and a pressure of from about 10 kPa to 5,000 kPa.

19. A process for the production of para-xylene comprising:
contacting a feed stream comprising methanol and toluene with a catalyst,
converting the feed stream to a product at reaction conditions, and
recovering a product comprising para-xylene,
wherein between about 10% and about 75% by weight of the catalyst comprises an improved MFI zeolite comprising in the calcined and ion-exchanged form a SiO₂/Al₂O₃ ratio of from about 100 to about 500, a heteroatom Q selected from the group consisting of boron, gallium, indium and iron, and mixtures thereof wherein the molar ratio of Si/Q is between about 2 and about 100 and having a low population of proximate framework aluminum sites characterized by an initial xylene selectivity of greater than 70% in a TM diagnostic test.

20. The process of claim 19 wherein the reaction conditions comprise an aromatic feedstock to oxygenate molar ratio of from about 0.5:1 to 10:1, a weight hourly space velocity (WHSV) of from 0.1 to 20 hr⁻¹, and a pressure of from about 10 kPa to 5,000 kPa.

21. The process of claim 19 wherein the product possesses a methyl to phenyl ratio of greater than about 1.75 and less than about 2.0.

22. The process of claim 19 wherein a methanol utilization is greater than 60%.
